(19) United States
(12) Patent Application Publication
(10) Pub. No.: US 2021/0268001 A1
(43) Pub. Date: Sep. 2, 2021

(54) USE OF MIOTIC CHOLINERGIC SUBSTANCES AND F2a PROSTAGLANDIN ANALOGUES FOR PREVENTION AND TREATMENT OF MYOPIA

(71) Applicant: Edson DOS SANTOS-NETO, San Paulo (BR)

(72) Inventor: Edson DOS SANTOS-NETO, San Paulo (BR)

(21) Appl. No.: 17/274,014

(22) PCT Filed: Sep. 5, 2018
(86) PCT No.: PCT/BR2018/050320

§ 371 Date: Mar. 5, 2021

(51) Int. Cl. A61K 31/5575; A61K 31/27; A61K 31/4425; A61P 27/10; A61K 9/00; A61K 31/407

(52) U.S. Cl. CPC A61K 31/5575; A61K 31/27; A61K 31/407; A61P 27/10; A61K 9/0048; A61K 31/4425

(57) ABSTRACT

The present invention relates to the use of miotic cholinergic substances alone or in combination with prostaglandin F2α analogues, for the preparation of a medicament compound for the prevention and treatment of myopia. It has been found that the use proposed in the present invention has resulted in the stabilization and reduction of the refractive degree, besides of the reduction of the elastance of the posterior pole of the eye and, consequently, of the CA/A ratio. Moreover, an improved visual acuity for all distances, with consequent improvement in contrast sensitivity and image sharpness, increased choroidal irrigation, and improved retinal cell action in the image receiving process was noted.

### FIELD OF THE INVENTION

The present invention relates to the field of medicine, and more precisely, to the field of ophthalmology, and describes a novel use of miotic cholinergic substances, alone or in combination with F2α prostaglandin analogs, for the preparation of a medicament for the prevention and treatment of myopia, by means of the activation of the parasympathetic-nitrergic system with consequent decrease of the Accommodative Convergence/Accommodation Ratio (AC/A), which is high in the beginning and progression of myopia, and therefore, is responsible for the genesis of myopia.

### BACKGROUND OF THE INVENTION

Myopia is the refractive error in which the visual image does not focus directly on the retina, but in front of it. It may occur (more frequently), by an elongation of the eye (axial myopia) and/or or by marked increase in the power of convergence of the cornea and crystalline. Consequently, there is difficulty on seeing the distance, the greater the degree of myopia. The word “myopia” comes from the Greek “small or closed eye”, since people with this condition often tighten their eyes to see better from a distance.

Myopic refractive error causes a very large burden on society and particularly on individuals, having a strong negative impact on their self-esteem, career choices and eye health. Glasses, contact lenses and surgery may help to correct it, but they do not address the underlying and causative defect: a slightly elongated eyeball, which shifts the focal point of the optic system slightly forward of the retina, rather than directly on it. In severe cases, the deformation known as pathological myopia (above 6 degrees) causes a stretch not only of the sclera, but also of internal ocular structures, such as the vitreous, choroid and retina, in a condition called myopic degeneration.

Myopia usually appears and accentuates during the individual's growth phase (up to 20 years), and is therefore closely associated with ocular growth (emmetropization). The eye, which is measures 17 mm at birth, grows 5 mm (30%) up to 6 years of age. During this period, the cornea loses about 4 diopters of convergence power and the lens will lose approximately 20 diopters of accommodation power. Thus, there is a progressive evolution towards emmetropia (normal vision), when the eye reaches approximately 23.6 mm (world average) by 13 years old. At 6 years, when the eye has about 22 mm, there is a minimum prevalence of myopes (about 2%). From 6 to 15 years, the mean ocular growth is only 1 mm (5%), but this small growth will be sufficient to rise the incidence of myopia to 15%, i.e., 7 (seven) times more.

At first, the adolescent notes a progressive worsening of the distance vision, especially at night (night myopia). When examined (under dilation) in the ophthalmology practice, any myopic grade is often discarded, or even small hyperopia is found. This is what we call pseudomyopia or parasympathetic tonus accentuated by the excessive use of near vision (computers, smartphones, etc.). At this stage, no myopic grade should be prescribed (which does not really exist), but in practice, the patient's parents press the teenager to wear glasses. There is a myth in modern society that not wearing glasses would increase the myopia degree.

There are also cases of late-onset myopia, which occurs after adolescence and do not usually progress to pathological myopia, as usually occurs in early-onset myopia. Whatever the type of myopia, the increasing number of short-sighted people in the world in the last decades has revealed a worldwide problem: an epidemic of myopia, which spreads throughout the countries of the five continents.

Myopia Epidemic

In the new millennium, myopia has gone from being just a visual disorder to becoming a serious public health problem around the world. In a challenging and highly disturbing article published in Nature, Dolgin warns of the epidemic spreading to new generations, from more than a hundred countries, related to the activities of the modern world, especially after the spread of computers and smartphones in homes of the whole society and reaching practically all social classes. According to the author, specifically East Asia has been dominated by an unprecedented increase in myopia. Sixty years ago, 10-20% of the Chinese population was myopic. Nowadays, in some places, it reaches up to 90% of Chinese teenagers and young people. In neighboring Seoul, for example, 96.5% of Korean men at the age of 19 are myopic (126).

Other parts of the world have also seen a dramatic increase in this condition, which now affects about half of young adults in the United States and Europe—twice the prevalence of half a century ago. “We are on the way to a myopia outbreak”, says Padmaja Sankaridurg, head of the myopia program at the Brien Holden Vision Institute in Sydney, Australia. This highly worrisome threat to the modern world has led to increased research to try to understand the causes of the disorder. Currently, it is believed that myopia is not limited to schoolchildren eager for extended reading. There is a consensus among the ophthalmic community that spending too much time indoor is putting children at risk by too much exposure to artificial light at near range.

“Half the population of Brazil and the world will be myopic in 2050 (127).” This is an alarming estimate of the American Academy of Ophthalmology (AAO), based on epidemiological data from a meta-analysis of 145 studies covering 2.1 million participants, released by a group of researchers from the organization. The document also states that 10% of the world population should have high myopia, a condition that is the cause of serious eye diseases. The meta-analysis estimates that, in 2020, the prevalence of short-sighted people in Brazil will be 27.7%, and in the US, 42.1%. The problem is expected to worsen by 2050, when 50.7% of Brazilians and 58.4% of Americans will be myopic, according to the entity's forecast. This means that, in the next 34 years, the incidence should increase 83% in Brazil, and 20% in the US.

Still according to this AAO survey, the scenario (which is currently catastrophic) is expected to worsen in the coming decades. In 2010, uncorrected myopia was estimated as the most common cause of distance vision impairment, affecting 108 million people and the second leading cause of global blindness. In recent years, the economic burden of this refractive error has been estimated at $202 billion annually, a figure that may be greatly underestimated.

Thus, there is a significant economic argument to eliminate uncorrected myopia and other refractive errors. In addition, the high incidence of myopia brings other social challenges: the higher the myopia, the greater the risk of ocular pathologies such as cataracts, glaucoma, retinal detachment and myopic macular degeneration, all of which may cause irreversible loss of vision. In some communities with a high prevalence of myopia, myopic macular degeneration was considered the most frequent cause of irreversible blindness. Myopic macular degeneration is also associated with 12.2% of vision impairment in Japan (approximately 200,000 people). About a fifth of college-age people in East Asia now have this extreme form of myopia; and half of that population should develop irreversible vision loss. In some Eastern countries, pathological myopia is already one of the three most common causes of monocular blindness.

According to a survey conducted in 1993, a myopic eye with an equivalent refractive error of −1 to −3 diopters has an increased risk of 4-fold of retinal detachment compared to a non-myopic eye; if the refractive error is greater than −3 diopters, the risk increases 10-fold. The data suggest that almost 55% of non-traumatic detachments (in eyes without prior surgery) are attributable to myopia.

So, neglecting the problem is by far the worst solution. An effective drug against the evolution of the epidemic and with few side effects would be a highly advantageous solution due to serious eye health problems arising in cases of pathological myopia.

The Causes of this Epidemic (Light and Nearwork)

Hereditary and environmental factors act directly in the genesis of myopia. Genetics is a risk factor for myopia in children, and the prevalence of myopia increases to 30% to 40% when they present two myopic relatives, reducing to 20% to 25% when they have only one relative and less than 10% when they have no myopic relatives. In addition, monozygotic twins present refractive errors more similar than dizygotic twins do. These data led to a hectic search for genetic markers and genes linked to myopia, a possible preventive solution to curb this epidemic. However, the marked number of genes found in this relationship hinders any kind of preventive measure.

Among environmental factors, there are strong consistencies in the literature that sunlight (UV) is a protective factor for developing myopia. Several studies in laboratory animals show consistent data of this relationship and associate dopamine release by retinal cells after exposure to the sun. This fact closely related to the modern lifestyle, where individuals live in closed rooms, with high limitation to sunlight exposure, something that stimulates the growth of the eye and induces the onset of myopia, especially in teenagers (130).

According to researches made by the present inventor, the incidence of UV radiation is the most important protective external factor to curb myopia evolution. It is important to stress that the enzyme acetylcholinesterase (AChE) is highly sensitive to UV radiation, which depresses its action (131). Thus, cholinergic drugs would have the additional function of chemically replacing sun deprivation in humans (inactivating AChE).

Nearwork is an English term that relates to near-prolonged activity (reading, computer use, etc.), which many researchers consider to be the most active environmental factor at the origin of myopia. It is the term that will be used in this text to describe near vision. One of the clearest signs of this factor in its genesis came from a 1969 study of people in Inuit, a region in the northern part of Alaska, whose lifestyle has been changing. Of the adults who grew up in this isolated community, only 2 out of 131 had myopic eyes. However, more than half of their children and grandchildren had the condition. Naturally, the genes could not explain this whole phenomenon. Genetic changes happen very slowly to justify such a rapid change to these high rates of myopia, which are also documented all over the world. “There must be an environmental effect that has caused the generational difference,” says Seang Mei Saw, who studies epidemiology and genetics of myopia at the National University of Singapore.

The recent increase in myopia reflects a modern tendency of children, in many countries, to devote more time to reading and studying close to computer screens and smartphones. This is particularly the case in East Asian countries, where the high value attributed to educational performance is driving children to spend more time in school and in their studies. A 2015 report of the Organization for Economic Cooperation and Development showed that teenagers (15-year-old on average) in Shanghai now spend 14 hours a week on homework, compared to 5 hours in the UK and 6 hours in the United States.

The researchers consistently documented a strong association between educational measures and the prevalence of myopia. In the 1990s, for example, they found that adolescent boys in Israel attending schools known as Yeshivas (where they spent their days studying religious texts) had far greater myopia rates than students who spent less time reading their books.

Convergence and Nearwork

The myopia epidemic raises doubts about the Helmholtz's Classic Theory of Accommodation (128). There is a group of researchers who do not find in it responses to the relation between nearwork and progression of myopia, in such worrying numbers. This group (including the present inventor) believes that too long and prolonged ocular convergence, with the joint action of extrinsic adductor muscles, would increase pressure in the vitreous chamber, causing axial stretching of the eyeball and consequently of myopia. Thus, we discard any kind of relationship between crystalline thickening and myopia genesis, as proposed by Helmholtz's Theory.

In 2011, the present inventor published a study suggesting a new approach to ocular accommodation, more consistent with the imaging findings used in modern ophthalmology (129). This more sophisticated “armed propaedeutic” has received great contribution with the advancement of technology in the examinations of sound and image developed in ophthalmology in recent decades. These modern exams definitively rejected the Helmholtz Theory, but it remains valid until nowadays since there is nothing better to counter it. However, classical theory is a major drawback that froze ophthalmology in time and prevents the development of new research in the various areas of vision. In the work published in 2011, the present inventor already warned about the shortcomings found in the classical theory of accommodation, but that still beacon and hinder all work done (related to nearwork and myopia), in the last 150 years.

According to Bayramlar et al., “axial elongation at near fixation (mainly due to increased vitreous length) may be due to the effect of accommodative convergence rather than accommodation itself. A great use of convergence, and not of accommodation, may be one of the contributing factors at the onset and progression of myopia in adults” (132). Bayramlar virtually disregarded Helmholtz's Theory. However, like so many other opticians, optometrists and ophthalmologists, it has not progressed and has not proposed a new solution to explain the mechanism of accommodation.

Briefly, comparing the two accommodation mechanisms (the classic Helmholtz versus the mechanism alleged in the present invention), the basics differences can be schematized according to FIG. 1. In (A), there is the classic crystalline thickening alleged by Helmholtz, which would increase the convergence power of the lens, with the accommodation the image in the retina for near vision. It does not explain the elongation of the eyes of large readers, where there is an intense and prolonged nearwork, a proven cause of myopia. In (B), the mechanism alleged by the present invention, justifying the origin of myopia in massive readers.

In the book “Ophthalmology in Drops” (which will be published by the present inventor), the various flaws of the Helmholtz Theory will be further detailed. For now, four errors of classical theory can be cited:

1. A typical voluntary action (accommodation for near), would not be performed by an involuntary smooth muscle (the ciliary muscle). In mammalian neurophysiology, the distinction between voluntary (skeletal muscle) and autonomic involuntary (smooth muscle) control is so important that many writers have observed the singularity of ocular accommodation, which is a voluntary function (commanded by the cerebral cortex) by a smooth muscle.

2. The Paradox of Lenses. This paradox, which was widely publicized in the last century, has always been the main argument against Helmholtz's Theory, with its critics Koretz and Handelman, Brown et al (133, 134). According to these authors, classical theory does not explain the incontrovertible fact that thickening of the lens occurs throughout human life (especially after age 40) without proper ocular myopization.

3. Accommodation-convergence Synkinesis. In Helmholtz's classic theory, several ocular structures closely related to the third cranial pair are merely supporting actors in the proposed accommodation mechanism. Thus, the Helmholtz's theory subjugated the classic triad of muscular synkinesis (miosis, accommodation and ocular convergence—universally linked to the accommodation mechanism). It also subjugated the fact that vision is binocular and the eye does not function as a photo camera alone. Moreover, it is not possible to associate, in the Helmholtz model, accommodation with the elongation and chronic deformation of the eyeball, as described in myopia. The accommodation is binocular and controlled by the two cerebral hemispheres. The incontrovertible fact is that, in 90% of human eyes, the control of the Emmetropization is symmetrical and generates approximate degrees of refraction in both eyes.

4. It does not explain the AC/A ratio. The main means of objectively measuring accommodation is through the AC/A ratio, which was created from the triad of muscular synkinesis (cited above), that is currently known to be the only parameter that has been shown to be altered in myopia (described below). Recalling also that Helmholtz followers have never convincingly explained the AC/A rate and mechanism of action of miotics in this clinical parameter, the main subject of the present invention.

A New Model for the Accommodation Mechanism

To stick to the text, it is necessary to describe a new model of the accommodation mechanism capable of incorporating all new concepts that will be developed in the following paragraphs, such as accommodative convergence, AC/A ratio, posterior pole hysteresis, complacency and elastance. The present inventor has studied this new model for several years. Due to its complexity, more details will be described in the book “Ophthalmology in Drops”. Briefly, we can divide the mechanism of ocular accommodation into two phases, with two consecutive reflex arcs (nerve impulses):

Phase 1. Convergence Reflex. Voluntary.

At this stage, the control is voluntary, controlled by the brain after visualization of the near object. Then, a contraction command to the ocular adductor muscles (MR, SR, IR and IO muscles—all innervated by the third cranial nerve) begins. The afferent nerve stimulus (initiated by the approximation of the object to the eyes) goes to the visual cortex through the Optic Nerve (II cranial pair) returning to the mesencephalon, where the oculomotor nucleus is located. Next, the neural stimulus is transmitted through the extrinsic efferent pathway of the oculomotor nerve, passing through the ciliary ganglion, to finally reach the motor plates of the adductor muscles. Simultaneous contraction of the extrinsic muscles causes convergence of the eyes and compresses the ocular wall, raising the intravitreal pressure (IVP) and pushing the lens towards the anterior chamber.

Phase 2. Accommodation Reflex.

(i) Involuntary. The displacement of the lens towards the cornea, pulling the ciliary zonule and moves the ciliary muscle forward. The traction caused by displacement of the ciliary muscle stimulates proprioceptive pressure receptors (Ruffini's corpuscles) in the ciliary body, generating a new afferent nerve stimulus to the mesencephalon. The main action of Ruffini's corpuscles is to avoid excessive traction and rupture of the zonular fibers, as well as the containment of the lens in the posterior chamber of the eye (keeping it behind the iris), thus preventing its prolapse to the anterior chamber. This is possible by contracting the circular and radial fibers of the ciliary muscle, which loosens the zonule.

(ii) The new nervous stimulus reaches the Edinger-Westphal nucleus and returns to orbit through the efferent fibers of the intrinsic portion of the oculomotor nerve, which synapse in the ciliary ganglion and stimulate the action of the sphincter muscles of the pupil and the ciliary muscle. The contraction of the sphincter muscles of the pupil, causing miosis, is a screen against lens forward shift of the lens. This reflex of the ciliary and sphincter muscles of the pupil are fundamental to control the IVP and the vitreous chamber stretching. The increase in IVP generates two opposing longitudinal forces: 1. in the anterior part of the eye pushes the entire block of structures (iris, crystalline and anterior vitreous) forward; 2. In the posterior part, it moves the macula towards the apex of the orbit, promoting the ocular elongation and the focus of the retinal image (nearvision), all controlled by the occipital cortex.

(iii) The contraction of the meridional fibers of the ciliary muscle also pulls the trabecular meshwork, facilitating the drainage of the liquids and decreasing the IOP (anterior chamber).

In short, the contraction of the adductor muscles will cause an increase in IVP, which shifts the lens towards the cornea. Sequentially, the afferent stimulus generated at the receptors (Ruffini) of the ciliary body go to the mesencephalon and return towards the ciliary muscle, passing through the ciliary ganglion. This stimulus will cause simultaneous contraction of the ciliary and sphincter muscles of the pupil, causing miosis and stretching of the vitreous chamber. According to this new model of accommodation, anterior lens displacement and ocular stretching, together, increase the focal length of the object in the retina, which allows its visualization in high definition in the plane of the macula. The basic voluntary command of this whole sequence of muscular actions arises in the convergence of the eyes, which increases the IVP and expands the axial diameter of the eyeball, that is, the accommodation for focusing the object in the macula. Tissue resistance to posterior pole expansion is a well-known physical phenomenon in animal physiology called elastance. This resistance force on the posterior wall of the ocular globe (choroid and sclera) becomes fundamental (according to research by the present inventor), since it represents clinically the AC/A ratio and can be easily evaluated in any ophthalmological office, as described follow.

The Relation between Accommodative Convergence and Accommodation

Physical optics teaches us that to focus an object at 33 cm (nearwork) a convergent power lens equal to 3 spherical diopters (+3.0 DE) must be used, that is, to accommodate 3 diopters. Since the nineteenth century, it has been known that a stimulus to alter the accommodation of the eye is accompanied by a change in the stimulus of ocular convergence, which is manifested by a certain deviation (phoria) in measurable binocular vision in prismatic diopters, on clinical examination. This mechanism is the accommodative convergence (AC). The ratio of accommodative convergence responsible for a certain level of accommodation (AC/A ratio) has also been known for a long time and expressed in this equation with prismatic diopters by spherical diopters (Δ/D).

The AC/A ratio can be evaluated in the ophthalmology practice in several ways. The gradient method is the most traditional and uses an apparatus (called synoptophore). It is the easiest method with the most reproducible results. Eyes are kept at a certain fixing distance from the object in space (by projecting a slide with the device), and then one or more spherical lenses of predetermined values (usually negative) are added. The phoria is then measured before and after inclusion of the lenses. The amount of convergence change caused by the diopter shift for the required accommodation is the classical AC/A ratio.

Experience has shown that the AC/A ratio is essentially the same for the same person at all distances of vision and for any change in accommodation stimulus. However, of great importance is the fact that the AC/A ratio is linear for most responses, according to changes in accommodative stimulus or, in other words, a change of unity in the stimulus to accommodation in general results in a corresponding specific amount of change in convergence. It is considered that the evaluation may, depending on the age, not be correct in cases where the requirement of accommodation for an individual is above his accommodative ability, especially after 40 years. There is also an “accommodative lag” (a delay in responding to the accommodative stimulus) in the myopes, and this may interfere with the extent or the way it is performed. In several studies, the gradient method proved to be reliable at a mean universal value around 3.50 Δ/D (with 1.25 Δ/D variation).

The CA/A rate has been used for decades in ophthalmology for the evaluation and treatment of accommodative strabismus. Hyperopic patients with accommodative esotropia present AC/A ratio at high levels, generally above 5.0 Δ/D. In these cases, the treatment of choice is currently the optical correction with the total prescription of hypermetropic error, to cancel the ocular deviation. In cases where AC/A is at a very high level (and keeps residual esotropia close by due to the high accommodative requirement), bifocal lenses may be required.

In the 50s, 60s and 70s of the last century, an abnormally high AC/A ratio could also be treated pharmacologically, an option for wearing glasses. Cholinergic drugs (also known as miotics) were widely used during this period, since they reduce these abnormally high rates in cases of accommodative esotropia. It was supposed that more potent miotics, such as acetylcholinesterase inhibitors (long-acting anticholinesterases, such as ecotiophate iodide), act directly on the ciliary body, facilitating transmission at the myoneural junction and reducing the central demand for the parasympathetic impulse. Miotics, then, would reduce the amount of convergence induced by accommodation and would counteract ocular deviation. It should be reminded that, according to the Helmholtz Theory, accommodation is the thickening of the lens, so, it makes no sense that miotics interfere with the action of the lens, decreasing the AC/A ratio and correcting the ocular deviation. This is one of the failings (among others cited above) that has never been adequately elucidated by Helmholtz's classical theory.

Thus, for decades, drugs were used in high concentration (with considerable side and systemic effects), without the proper knowledge about their mode of action in the ocular tissues. That is, in reality, there has never been a minimally enlightening explanation, both for the significance of the AC/A ratio and for the action of miotics on accommodative strabismus. In recent years, the synoptophore is no longer used in most ophthalmological offices and has become obsolete. Currently, the AC/A ratio is used only in specialized strabismus services of university institutions; and if no new fact occurred, the AC/A ratio would be disregarded even in ophthalmology manuals.

AC/A Ratio and Myopia

Almost forgotten in ophthalmology books, the AC/A ratio became of utmost importance in prior diagnosis, treatment and follow-up of myopia progression, as it is shown by the ingenious multi-center study (The Collaborative Longitudinal Evaluation of Ethnicity and Refractive Error—CLEERE—Study), devised in the United States almost three decades ago. As it can be seen in FIG. 2, over a period of 10 years (on annual visits), the AC/A ratio increases exponentially at the beginning and after the evolution of myopia.

This is surprising and promising data for the prior diagnosis and follow-up of myopic children. The study provides a direct correlation between the AC/A ratio and myopia growth. It was already expected that accommodation related to myopia progression. But what is the meaning of accommodative convergence linked to this progression? Answering this question is the same as filling in the puzzle involving the genesis of myopia in nearwork. A difficult and complex question with no clarifying answer to date. To solve this problem, it should firstly consider the conclusion of the study of Bayramlar et al. previously cited, as well as the study of the present inventor (new accommodation model) that links the convergence of eyes to ocular stretching.

CLEERE was conceived (in 1997) as a project derived from the Orinda Longitudinal Study on Myopia (OLSM). Orinda is a city located in the US state of California and the study with local school students was started in 1989 (thus, in almost three decades) to investigate normal eye growth and the development of myopia in more than 1,200 children of age school. From 1997 onwards, it was divided into three parallel phases, being carried out until today with the name of CLEERE. Phase 1 investigated additional factors that may predict the onset of juvenile myopia (accommodative function, peripheral refractive error, intraocular pressure and school achievement). Phase 2 compared optic ocular components and refractive error profiles of other ethnic groups with the predominantly Caucasian Orinda database. Phase 3 conducted DNA-based studies on prevalent myopes of OLSM and their families to use these phenotypically well-characterized children and a panel of candidate genes in the search for evidence of genetic factors. In Phase 1 of CLEERE, the annual examinations of the children of the Orinda Union School District were continued in the age group of 6 to 14 years. Measurement of accommodative response, accommodative lag, phorias, response AC/A ratio, peripheral refractive error and intraocular pressure (IOP) were added to the existing protocol.

In the CLEERE study, 3,493 patients (since 1999) are still being examined in four clinical centers. African American, Hispanic, and Asian children are examined annually for at least four years. Exams include visual acuity, refraction by a variety of methods (cycloplegic and dynamic autorefractor test), cover test with fixation at distance or near, assessment of accommodative response with autorefractor, AC/A ratio measurement, video-facometry, peripheral refraction, and A-ultrasonography.

Several papers have been published in high impact scientific journals from these studies. Two in particular brought enlightening results, from teenagers who developed myopia. In 2017, Mutti et al. published a complete study presenting the results shown in the FIG. 2 (135). In 2010, the same author had already evidenced the direct relationship between accommodative convergence and myopia, and that myopic patients frequently had esophoria. In the 2010 study, the authors also found the increased AC/A ratio (statistically significant).(136) Adjusted for age, the AC/A ratio was higher in myopes (6.39 Δ/D), intermediate in emmetropic eyes (3.94 Δ/D) and lower in hypermetropic eyes (3.40 Δ/D) (FIG. 3). The stimulus AC/A ratio did not vary with the refractive error. Once adjusted for the refractive error, the response AC/A ratio did not change with age either. In non-myopic children with a response AC/A ratio of 5.84 Δ/D (or greater), the risk of developing myopia increased within 1 year by 22.5 fold. In a sub-sample of children without myopia who had refractive errors of less than +0.75 D with an AC/A response ratio of 5.84 Δ/D or higher, the risk of developing myopia within 1 year increased 3.21 fold.

Other studies investigating refractive error and the AC/A ratio agree that myopic teenagers have higher AC/A ratio than other refractive groups. A study conducted by Flom and Takahashi, in the early 1960s, already demonstrated it (137). This shows the magnitude of the finding described in the present patent. Other studies also confirm that early-onset myopia (as well as late-onset myopia) have higher AC/A ratio, suggesting that an increase in AC/A ratio may also predict the onset of myopia in adults. Retrospective data from myopic children show that the increase in esophoria occurs approximately 5 years before the last examination (when the child is still emmetropic) and becomes more convergent for approximately 1 year after the myopia onset. These data are extremely relevant and (taken together) they confirm the direct relationship between accommodative esotropia, myopia and high AC/A ratio.

Although the evidence of an association between myopia and esophoria is not entirely conclusive, it is positive. Data still gathered in CLEERE show that the more the myopia is corrected (hypercorrection), the greater is the resulting AC/A measured after the new prescription. That is, in order to provide better vision with glasses or contact lenses, optometrists and ophthalmologists cause more severe myopia in their patients and aggravate this epidemic. A better solution would be to use bifocal lenses for children, as it is done in East Asia.

A New Biological Marker

The CLEERE study is arguably the most comprehensive study carried out on myopia in the history of ophthalmology. The results unequivocally highlight the accommodative convergence as the decisive factor in the genesis of myopia. It is a significant and illuminating advance for many doubts that are still unanswered in the twentieth century. The main one is why the prolonged use of computers aggravates myopia? With all of the foregoing, it can be stated that prolonged convergence in front of a computer can permanently lengthen the eye and cause myopia.

In the absence of conclusive results in the early stages (OLSM), in the subsequent phases of the original project, extended genetic and racial research. However, it confirmed only a myriad of factors in the genesis of myopia. It would require customary treatments for each child, impossible to be carried out in the midst of an epidemic. Even in the medical literature, tens (perhaps hundreds) of gens were evidenced with probable connection with myopia. However, nothing compares to the AC/A parameter, which is easy to perform in an eye clinic and signals the actual onset of refractive deviation. It is a new biomarker to aid in the prevention and treatment of myopia. Before using this marker, it is important to know the ocular physiology that relates myopia to the convergent deviation, described below.

Of the muscles involved in the synkinesis of accommodation, only the extrinsic muscles are voluntary, which coincidentally are responsible for ocular convergence. Thus, it is possible to associate the origin of myopia with the nearwork, confirming the suspicion of Bayramlar et al., “A great use of convergence, and not of accommodation, may be one of the contributing factors at the onset and progression of myopia in adults”.

Ocular convergence is the result of a joint action of the adductor muscles—the rectus muscles: medial (MR), superior (SR) and inferior (IR)—in addition to the inferior oblique muscle (I0) (which in the reading position, becomes an adductor). All of them are extrinsic (voluntary) muscles and innervated by the extrinsic portion of the oculomotor nerve. Therefore, they are also part of the synkinesis of accommodation. This integrated action of this muscle complex promotes compression throughout the wall of the eyeball and elevates intravitreal pressure (IVP), lengthening the eye and providing accommodation. This explains the AC/A ratio, where the IVP is nothing more than a representative parameter of the accommodative convergence effort. Thus, from now on, the IVP will be represented by the letter P and its variation by the symbol ΔP, that is, AC=ΔP.

On the other hand, the ocular stretching that (according to the novel accommodation model) physiologically represents the variation of the volume of the vitreous chamber (or axial length) will be expressed by the symbol ΔV, that is, A=ΔV. Thus, the AC/A ratio=ΔP/ΔV. This P×V ratio has been already well studied in the physiology of other human tissues (such as the lung and brain) and is known as elastance (ε). The description of the entire elastance component will be further detailed in the book “Ophthalmology in Drops”. As the AC/A ratio became of paramount importance in relation to the onset of myopia, we will partially describe it herein.

The Passive Components in Accommodation

Brown et al., in the late 1990s, published one of the most important papers on Emmetropization, the mechanism that leads most human eyes (80%) to emmetropia at the end of ocular development, at the age of 13. It is an exceptionally high percentage compared to the eyes of other lower mammals on the zoological scale, where emmetropic eyes are the exception rather than the rule. According to Brown, this fact could only be explained by the presence of passive components and mainly active during the process of accommodation and Emmetropization.

Thus, we can divide these components into active and passive. The active components, as already explained, refer to the muscle groups that make up the classic trilogy of oculomotor synkinesis; the passive components are those dependent on the pressure of the vitreous chamber (IVP) and the elasticity of the posterior wall of the eye (Hysteresis):

1. Active components (muscles that are part of the ocular synkinesis)
(i) Intrinsic Muscles—Ciliary Muscle (accommodation) and Iris Sphincter Muscle (miosis).
(ii) Extrinsic Muscles—Adductor Muscles (convergence).

2. Passive components (related to IVP and hysteresis)
(i) Elastance
(ii) Complacency

The active factors are of decisive importance in ocular stretching, but the passive components are fundamental in emmetropization (formation of an emmetropic eye), as well as in the origin of the refractive deviations, mainly myopia. The scleral thickness is of relative importance in these cases and will not be taken into account here because it can be altered by the action of the choroid and of myofibroblasts (MFs). In this novel model of accommodation proposed in the present invention, the elevation of IVP is the stimulus that triggers a series of actions that will provide ocular stretching and deserve more attention.

Choroid MFs could be included as active components of the accommodation, but (by its action as a choroid and sclera relaxant) it will be included among the passive components. It was extremely necessary to describe all these components (active and passive) to explain the action of the medications used in the treatment and prevention of myopia.

Hysteresis, Complacency and Elastance

A hysteresis is a well-studied phenomenon in classical physics and refers to the tendency of an object to conserve its properties in the absence of a stimulus that can deformed it, or even an ability to preserve its shape after the withdrawal of a stimulus which deformed it. In other words, in biology, hysteresis refers to the elasticity of tissues and their tendency to deform or return to their original state after the force that distorted it has ceased. In the posterior, pole of the eye, the greater the hysteresis of choroid-sclera, the greater its elasticity. The stimulus that deforms this set is the intravitreal pressure (IVP), which negatively influences the hysteresis, i.e., when the vitreous wall is inflated by the increase of IVP, there is a consequent reduction in the elasticity and hysteresis of the tissue, or vice-versa.

Two observations are important here:

1. Intravitreal pressure (IVP) should not be confounded with intraocular pressure (IOP). They are parameters used in different regions of the eyeball; IOP is used in the front portion and IVP is used in the posterior portion of the eye.

2. Hysteresis should not be confounded with elastance; hysteresis is a measure of the tissue elasticity and elastance, on the other hand, is the resistance of the tissue to its expansion.

Besides IVP, hysteresis of the posterior wall of the eyeball is also related to the presence of collagen in the local tissue structure, as well as to the presence of MFs in the choroid, which influences the local elasticity. In hollow organs, such as the eyeball, the elasticity of tissues plays an important role in physiology. In the stomach, bladder or even blood vessels, pressure on the walls of the organ will cause volume expansion in a proportionally direct ratio, which can be demonstrated in the graph curve of FIG. 4.

The graph of said figure can be analyzed from its two components: complacency and elastance. In this updated model of stretching, the Pressure and Volume (P×V) ratio is a direct consequence of the actions of the muscles in the eye walls. The great practical innovation of this model is that it allows a mathematical correlation in this P×V ratio (which is known as elastance in human physiology) and obtained from an ophthalmologic clinical evaluation: the AC/A ratio.

The graph of FIG. 4 refers to lung hysteresis, an important organ that serves as a comparative example with the eyeball. Complacency and elastance are two ways of evaluating lung distensibility. Lung elastance (ε) corresponds to the resistance of the tissues to the alveolar expansion when inflated (during inspiration) determined by the entrance of air in the airways; it is the conceptual opposite of complacency (C), which corresponds to the ease with which the lungs accommodate air volumes (ΔVpulmonar) and return to the resting state, by decreasing the expiratory pressure (ΔPtranspulmonar). These variables are determined in the pressure-volume curves and expressed by the equation: C (L/cm² de H₂O)=ΔVpulmonar/ΔPtranspulmonar (transpulmonary pressure is the difference between pressure, at alveolar level, and pressure in the pleural space). Briefly, complacency is the inverse of elastance: ε=1/C, and vice-versa.

Complacency and elastance always expressed as positive values, since the variation of volumes and pressures, at the pulmonary level, occur in the same direction. The lung is not a tissue with perfect elastic characteristics: it has a heterogeneous structure and a liquid-air interface at the alveolar level, which generates great surface tensions. Thus, lung pressure-volume curves are complex. The insufflation component is different from the emptying component, and a pressure-volume cycle with large discontinuity between its two phases (as shown in FIG. 5) is designed. The designation that a physical phenomenon receives, in which pairs of values of two variables (which are function of each other and do not repeat themselves when they vary in one direction and in the opposite direction) is hysteresis, as it is shown in the graph.

Ventilation is a mechanical and biological activity that clearly evidences hysteresis. Collagen seems to be responsible for limiting lung distension as lung volumes increase to values near the maximums, similar to that occurring in the posterior wall of the eye, under pressure. There are about 200 million alveoli in the lungs of an adult human being, which means a surface area of approximately 70 m². Throughout this area, an air-liquid interface generates surface tension. Surface tension is a key element of respiratory mechanical properties. The example of the lung brings us very close to the eyeball. There is an interesting pathophysiological alteration in the alveoli of their hysteresis (similar to what occurs in the posterior wall of the eye), which can be used as a clinical example of drug interference in tissue hysteresis. The surfactant substance is a complex organic substance with phospholipid surfactants, whose action, in general terms, is the reduction of the surface tension. Much of the variability of the surface tension, between insufflation and emptying, seems to be due to the phospholipid molecules. Thus, in the same way that the surfactant is used in the lungs to improve tissue hysteresis, in the eye the, use of miotics (via nitric oxide) has a similar proposal.

Complacency and Elastance of the Eyeball

In ocular physiology, the present invention is evidencing two new physical terms:

(a) Complacency, which is a measure of the resistance of an organic tissue to the retreat to its original dimensions with the removal of a compressive or distensive force;

(b) Elastance, a term inverse to compliance, which is the resistance to expansion of the vitreous chamber volume, limiting the increase in axial length in relation to the increase in IVP. The equations that relate these parameters (C=ΔV/ΔP and ε=ΔP/ΔV) are represented in the graph of FIG. 5.]

As these concepts are novel in the art, the subject will be probably discussed in the future, with further complementary research. However, the present inventor believes that the graphic 5 design will be entirely useful to explain the various pathophysiological changes that are routinely observed in the ophthalmology practice and that will be reviewed below.

As is well known in the study of corneal biomechanics (keratoconus), elevation of IOP is inversely proportional to tissue hysteresis. According to the present invention, the biomechanical conditions are similar in the posterior pole, but as several studies indicate, the vitreous pressure (IVP) varies with greater frequency. During accommodation, the IVP rises, while the trabecular opening reduces the IOP (in the anterior chamber, measured by the applanation tonometer). The increase of IVP during accommodation unfortunately cannot be measured by any clinical examination currently known. Therefore, the pressures in the vitreous and in the anterior chamber are of different and independent values and it is not possible to determine them simultaneously “in vivo”.

IVP elevation is a reflex reaction to accommodative convergence (as stated above) and can be represented by the AC/A ratio, or more specifically, by accommodative convergence (AC). Van Alphen demonstrated a practical example between the IVP and AC relationship (138). In the article, the elevation of IVP through a cannula with infusion of saline solution directly from the vitreous chamber, the researcher caused a choroidal stress (denominated by van Alphen) and the persistent ocular stretching, that is, the IVP caused a change in hysteresis and deformed the sclera. Van Alphen determined an IVP value (15 mm Hg), where there was ocular stretching in all eyes studied, above which there was choroidal stress. In FIG. 5, the beginning of this phase is represented by point B. In summary, elevation of IVP causes ocular stretching, and then, axial myopia. It would also stimulate biosynthetic changes in the sclera or choroid (perhaps in the lamina fusca sclerae, which lies between the sclera and the vascular choroid). The result of increased IVP is scleral remodeling (creep) by the action of fibroblasts on the sclera, reducing the synthesis of proteins and proteoglycans, and stiffening the sclera.

Sclera and Choroid

The animal eye is very similar to a soccer ball, presenting two spheres that form its original shape. The innermost is the choroid (elastic), while the outermost is the sclera (more resistant). The sclera (sclerotic) has a consistency that varies throughout life. In the first years of life, the white layer surrounding the eye is as elastic as the choroid. Only after the age of 30, the sclera begins to exert resistance to choroid deformation and its stretching. It is a prelude of presbyopia. At 30 years, the chance of myopia or myopic progression is about 10%, versus 50% at 20 years. Thus, it is around the age of 30 that the indication for refractive surgery of myopia is more adequate, when there is stabilization of ocular growth, evidencing the relationship between sclera hardening with ocular aging.

It is demonstrated that the local choroid determines the thickness of the sclera that covers it, since the choroid produces the cell factors of tissue growth, controls the biosynthetic reactions, the activities of sclera tissue remodeling and, finally, the shape and size of the eyeball. The choroid is a fibrovascular tissue, but with a clear predominance of its vasculature. Among other human tissues with similar characteristics, the choroid might be functionally compared to the cavernous bodies of the penis. The cavernous body filled with blood makes the tissue more elongated, warmer and more elastic (increasing its hysteresis). Similarly, the smooth muscles of the choroid (including the muscles of the walls of the blood vessels) present innervation similar to the cavernous bodies; that is, parasympathetic innervation, associated with nitric oxide (ON), named nitrergic innervation (parasympathetic-nitrergic system) (140). The human choroid also contains nonvascular smooth muscle cells, also known as myofibroblasts (MFs). It has intrinsic choroidal neurons, mainly behind the macular retina, which can control these MFs and can also modulate choroidal blood flow, which is very similar to the penis tissue.

This complex structure explains the various coordinate functions of the choroid: (i) its vascularization is focused primarily on the supply of the external retina and the impairment of the oxygen flow from the choroid to the retina can cause age-related macular degeneration; (ii) choroidal blood flow (proportionally too high for a living tissue) can also cool or heat the retina and sclera, depending on the amount of heat that arrives at the site, by exposure of the retina to sunlight or artificial light; (iii) In addition to its vascular functions, the choroid contains secreting cells, probably involved in the modulation of vascularization and sclera growth.

In laboratory animals, it is easy to demonstrate that the choroid reacts to the apposition of negative or positive lenses, known as defocus. These changes in choroidal thickness move the retina forward and backward, bringing the photoreceptors to the focus plane of the image presented to these eyes. In humans, the thinning of choroid with the apposition of negative lens is observed, and inversely, by the thickening that occurs when positive lenses are used and these momentary changes define what will occur with the sclera if the lens persists for a prolonged period. The biosynthesis of this material on the choroid and sclera (easily proven in lower animals and possibly in humans) is a fundamental data to understand the genesis of myopia. Thus, transient ocular stretching, in nearvision, could become definitive. That is, a prolonged nearwork triggers a series of reactions and long-term scleral remodeling, an initial and determining step in the origin of a myopic eye.

If in the model of Helmholtz classical accommodation the crystalline was the protagonist in the command of actions, in the current model described by the present inventor, this role of protagonist is carried out by the choroid. It governs the accommodation through its stretching and subsequently produces the scleral growth factors that will give definite shape to the eyeball. However, the current ophthalmological literature has shown us a decisive factor in the precise control of ocular stretching (accommodation): a network of MFs in the choroid.

Myofibroblasts of Choroid and Nitric Oxide (ON)

MFs have been extensively studied in the last two decades (139) and probably are involved in the largest findings of recent times in ocular anatomy, but their function in ocular stretching and emmetropization has not yet been adequately clarified. They are present in the innermost portions of the sclera (posterior wall of the eyeball) and are directly linked to MFs of the suprachoroid. However, they are more numerous and active in the choroid, where they are also known as nonvascular smooth muscle cells (NVSM) and may play a major role in stretching of the posterior wall of the eye. MFs are activated by the nitric oxide (NO) of the parasympathetic-nitrergic system (NS), synthesized mainly by the endothelial cells of the blood vessels and choroid macrophages. MFs form a true elastic network in the posterior pole of the eye, which give oval shape to this region, when activated in the ocular stretching. FIG. 6 shows this NVSM network.

During embryonic development, the MFs belonging to group B (FIG. 6) can sometimes be observed in the twentieth week of pregnancy. However, a network such as of adult humans was only first seen in a 6-year-old human donor eye. MFs of all groups can be characterized histochemically as true contractile cells. Thus, this elastic network can be compared to a cell syncytium that, when contracting, promotes: (I) the thinning of the choroid; (ii) empties the large suprachoroid gaps; (iii) allows vasodilation of the choroidal vessels (heating the local tissue and softening the outermost local sclera) and (iv) allows the transmission of all this elasticity to the entire elastic set from the ends of the ciliary muscle, which governs this contraction, in the anterior part of the NVSM network. The perivascular location of MFs may have a function similar to that occurring in the cavernous bodies of the penis, imprisoning the blood in the veins and inflating the choroid during accommodation.

The function of MFs of the choroid and sclera have not yet been fully defined, but due to the location and cell characteristics, three primordial actions can be deducted:

1. MFs actively control ocular growth from the age of six years. They form a restraining elastic network that alleviates the elevation of IVP, preventing the indefinite growth of the eye. However, since they are responsive to ON, the relaxation of this smooth muscle network allows ocular stretching during accommodation, thus becoming a mechanism of fine adjustment of the axial length of the eye in the accommodation with primordial function in emmetropization.

2. NO has an important action in the remodeling of wounds and can have the same function in the sclera. It activates the fibroblasts in the production of collagen, enriching and remodeling the sclera (scleral creep). It is possible that scleral remodeling can occur at any age, even after 40 years.

3. The NO action reduces the elastance of the posterior pole. During accommodation, acetylcholine contracts the ciliary muscle in the front portion of the eye. In the posterior pole, by the action of the nitrergic-parasympathetic, it activates and relaxes the MFs, in a sequence of chemical reactions described in FIG. 7. It also promotes a complex movement similar to the peristalsis of the digestive system (also innervated by the parasympathetic), but which induces ocular stretching in the eye.

Cholinergic Receptors

As previously described, the autonomic parasympathetic system commands practically all the physiological autonomic actions of the eyeball. In postganglionic neurons of the parasympathetic nervous system, the main neurotransmitter is acetylcholine. Analogously to the sympathetic system, some differences in the receptor of the target cells also occur in the parasympathetic system. Primarily, they are divided into two major groups: muscarinic receptors and nicotinic receptors. These names are because the muscarinic substance (a fungus) activates only the muscarinic receptors. Nicotine (tobacco) only activates nicotinic receptors. Acetylcholine activates both.

A cholinergic receptor (AchR) is an integral membrane protein that generates a response from an acetylcholine molecule. It is found mainly in neuromuscular endings, both in the central nervous system and in the peripheral system. Like other transmembrane receptors, the acetylcholine receptor is classified according to its pharmacology, that is, according to the relative affinities and sensitivity of different molecules. Although all acetylcholine receptors, by definition, respond to acetylcholine, other ligands may bind to them.

Nicotinic receptors are found primarily at the folded edges of the neuro muscular junction and in the postsynaptic ganglia of the parasympathetic system, and are activated when acetylcholine is released at the synapses. The diffusion of Na⁺ and K⁺ through the receptor causes depolarization and the opening of voltage-regulated sodium channels, allowing the onset of action potential and, finally, muscle contraction.

Muscarinic Receptors

Muscarinic acetylcholine receptors are part of the G protein-coupled receptor superfamily (a class of membrane proteins involved in the transduction of cell signals) and activate the ion channels through a cascade of chemical reactions, mediated by a second messenger. We find muscarinic receptors on all target cells of the parasympathetic system, as well as on target cells in sympathetic postganglionic neurons that are cholinergic. At least 5 muscarinic receptor subtypes are described, M1 to M5. The action they take depends on their location, as well as on the type of G protein to which they are coupled:

(i) M₁, M₃ and M₅—Receptors coupled to the G_{q/11} protein. Its activation promotes the phospholipase C activity (PLC), causing as a rule an increase in the function of the organ to which they are coupled. Two mechanisms are important, described in the following examples:
In the smooth muscle cell—Activation of the G_{q} protein induces increased PLC activity, which degrades membrane phospholipids by increasing the cytoplasmic concentration of inositol triphosphate (IP3) and diacylglycerol (DAG). IP3, by its turn, leads to the release into the cytoplasm of calcium (Ca²⁺) sequestered inside the cell, inducing muscle contraction (actin/myosin interaction). DAG has, among other effects, a role in the late (tonic) phase of the response.
In the endothelial cell—The apparent paradox posed by vasodilation mediated by muscarinic agonists contravenes the expected vasoconstriction by action on vascular wall musculature, which can be explained by the action of endothelial cells. Activation of the G_{q} protein induces an increase in the cytosolic concentration of Ca²⁺ by the same mechanism described above. In the endothelial cell, which did not have a contractile mechanism, the role of Ca²⁺ promotes, instead of binding to calmodulin, activation of the synthesis and release of nitric oxide (NO). This gas diffuses easily into the vascular musculature, where it will induce an activation of guanylate cyclase and consequent increase in intracellular concentration of cGMP, a potent smooth muscle relaxant. (FIG. 7).

(ii) M₂ and M₄—These receptors are coupled to an inhibitory Gi/O protein, which acts to inhibit adenyl cyclase. Keeping the role of cyclic nucleotides in muscle in mind, cAMP and cGMP are relaxing, with the exception of cAMP in the heart, where its effect is stimulating.

Muscarinic Receptors and the Nitrergic-Parasympathetic in the Eye

In the eye, some subtypes of muscarinic receptors have been identified in various tissues and play critical roles in ocular physiology. The ciliary body and iris sphincter muscle express all five subtypes of receptors, the predominant subtype being M₃. The parasympathetic stimulation of these tissues classically results in accommodation, altered aqueous flow and contraction of the iris sphincter muscle. Others M receptors are found in various ocular structures and even considering the main action of the M₃ receptor on the eyeball, the acetylcholine coupling mechanism in this receptor is more complex than what is seen in other organs. Thus, the parasympathetic ocular system would act in two different and to some extent paradoxical ways: sometimes stimulating the muscle contraction, and sometimes promoting the muscular relaxation. Thus, it is possible to divide the ocular parasympathetic into two subtypes: (i) traditional parasympathetic, which promotes the contraction of the effector muscle (ciliary and sphincter muscles of the pupil); (ii) nitrergic-parasympathetic, an effector muscle relaxant, through the action of nitric oxide (choroidal and sclera MFs) (141).

From the observations above, it can be defined that, in the eyeball, the nitrergic system is the main agent of the accommodation and NO (via acetylcholine) assumes a function similar to the surfactant substance of the lungs. In addition to its function in the muscle relaxation of the network of MFs in the posterior pole during the cholinergic parasympathetic action in the accommodation, NO facilitates ocular stretching (making the choroid more elastic).

In the modern and summarized view of accommodation, NO is the final product of the action of the ocular parasympathetic and acquires three primordial functions from its activation:
1. Relaxation of MFs and ocular stretching during accommodation
2. Vasodilation of the choroid, increasing its blood flow and improving the oxygenation of the photoreceptors, promoting a clear vision in high definition.
3. Activates the formation of fibroblasts (cells that produce collagen) and remodel the sclera, during the development of refractive errors (scleral creep).

NO is a key signaling molecule in the regulation of brain and ocular blood flow. At low concentrations, NO produced in various brain sites (after some type of injury) is a potent cerebral vasodilator, increasing blood flow (140). However, in high concentrations, NO has a high toxic activity. In the corneal epithelium something similar occurs with on topical instillation. Low concentrations of NO (1 nM to 1 mM NaNO₂) significantly increased the viability of epithelial cells, 6 hs after its application. However, a high concentration of NO (100 mM NaNO₂) resulted in a cytotoxic effect over the same period. Thus, it is possible to explain the antagonistic action of miotics in the choroid and to justify the use of miotics only in low concentrations for the treatment and prevention of myopia: (i) in low concentrations, the ocular stretching decreases; (ii) however, in high concentrations, the accommodative tonus increases and accelerates the progression of myopia. As it was exposed, the miotics activate NO and relax the MFs in the posterior pole. However, in the front portion of the eyeball, they contract the ciliary muscle and sphincter of the pupil, where the NO does not act. Therefore, opposing actions in the smooth eye muscles.

Miotic Cholinergic Drugs

Miotics belong to a class of drugs well known in ophthalmology. Pharmacologically, they are part of the cholinergic category, which have been studied for centuries in human eyes. The earliest cholinergic representatives used in medicine were atropine (exceptionally a mydriatic drug, a function opposed to miotics). It is also called belladonna due to the mydriatic and sensual effect in women (middle age), atropine blocks the acetylcholine receptors on the motor plate of the iris sphincter muscles, thus inhibiting the cholinergic action of the receptor and thus being a potent ocular dilator. Atropine and its mydriatic derivatives also block the ciliary muscle and are used in refractive exams due to its cycloplegic action.

Separately, the miotic cholinergic drugs are listed in two groups of drugs: (i) cholinergic agonists: in this group, we find the drugs that have acetylcholine-like action (but more potent) and that bind to the cholinergic receptors, remaining in the active sites of smooth muscles for longer. They, therefore, have a more potent and prolonged action than acetylcholine. The classic examples are pilocarpine and carbachol, with a period of action of approximately six hours. Pure acetylcholine has a very brief effect and disappears from the motor plate of the activated muscle in a few minutes; (ii) acetylcholinesterase enzyme inhibitors: This group is subdivided into reversible and irreversible inhibitors of the enzyme acetylcholinesterase, which degrades the acetylcholine in the motor plate where the cholinergic receptors of the parasympathetic system are found. They have a longer action time than the previous group. (ii.a) Reversible inhibitors (ex. physostigmine, neostigmine, edrophonium, rivastigmine, donepezil and pyridostigmine) has an action time of one to two days activating the cholinergic receptors with greater potency. (ii.b) Irreversible acetylcholinesterase inhibitors are the most potent miotics with the most frequent side effects. The action of these cholinergic can last for several days or weeks, depending on the concentration. As eye drops, the best-known representative of this group is phospholine iodide, used as an ocular hypotensive, or in accommodating strabismus for decades.

All cholinergic substances, for centuries, are used in medicine (via oral, muscular or intravenous routes). Especially in ophthalmology, eye drops have been used for more than a century as ocular hypotensive agents. They form a group of substances that act primarily on the parasympathetic system (partially in the sympathetic nervous system), activating muscarinic cholinergic receptors and mimicking or potentiating the action of acetylcholine, causing ocular miosis, a clinical sign that typifies the group as miotic. Some authors include in this group mydriatic drugs, since they bind to the same parasympathetic acetylcholine receptors (motor plate), but block them. We can then subdivide the miotic cholinergic into 3 groups:

(i) Direct acting cholinergic agonists—direct acting drugs on the acetylcholine receptor, activating it and mimicking (or potentiating) the action of this neurotransmitter:
(a) Acetylcholine derivative esters: Betanecol, Carbacol and Metacholine;
(b) Alkaloids: Muscarine and Pilocarpine;

(ii) Indirect Action Drugs—They have no direct action on cholinergic receptors, but inhibit the enzyme that can destroy acetylcholine, therefore acetylcholinesterase inhibitors or anticholinesterases. They provide longer acetylcholine action time and greater potency than the natural neurotransmitter.
(a) Reversible: Edrophonium, Neostigmine, Physostigmine, Pyridostigmine, Rivastigmine, Donepezil, Galantamine and Memantine.
(b) Irreversible: Malation, Paration and Fosfoline Iodide;

(iii) Mydriatic cholinergic—These agents block the actions of the parasympathetic system, having ocular activity similar to the sympathomimetic drugs, with emphasis on mydriasis and cycloplegia.
(a) Atropine-like drugs: Homatropine, Tropicamide and Cyclopentolate;
(b) Atropine group: Atropine (Belladonna), Hyoscine (scopolamine) and Pirenzepine;

Ocular Hypotenive Drugs

Ocular hypotensive have been used in the past in an attempt to slow the progression of myopia, but without success, including inhibitors of carbonic anhydrase and timolol maleate. The use of hypotensive agents was an attempt to prevent the evolution of myopia to pathological myopia (for various reasons) without any scientific basis. It is not the case here to explain the failure of the procedures, but there are few papers on the subject in recent years due to these discouraging results.

Three observations are important here:
1. Miotics were never used for the treatment of myopia, on the assumption that they could cause accommodation spasm and (conversely) increase the myopia grade.
2. Atropine (in various concentrations) has been used insistently to slow the progression of myopia, with dubious results. It has not been abandoned yet by the inexistence of any other minimally effective therapy when it comes to myopia.
3. Miotics are substances known to have limited effects on IOP reduction. The same occurs with Beta-blockers and Carbonic Anhydrase Inhibitors, which have already been used to treat myopia.

Prostaglandins (Pgs)

The use of ocular hypotensive to delay the progression of myopia occurred at a pre-prostaglandin stage. Launched on the market in the late 1990s, the Pgs group is considered the most effective among ocular hypotensive drugs and its side effects are already well established in the medical literature. They are also the drugs most used in the treatment of glaucoma in recent years. However, its mechanism of action is still somewhat unknown, although it is assumed that Pgs improve drainage of the aqueous through the supra-choroid by facilitating the transport of fluid through the fibers of the ciliary muscle.

Currently, prostaglandin eye drops are used with a daily dosage since it has a half-life of 24 hours, similar to anticholinesterases, thus facilitating their joint formulation. As eye drops prescription, in this category of prostaglandins, the most known substances are latanoprost, bimataprost, travaprost and unoprostone.

There is no description in literature (to date) on the use of Pgs alone in any type of myopia therapy. However, the present inventor assumes that the action of Pgs in this type of treatment (even if alone) is a highly effective adjuvant drug, not only as an ocular hypotensive agent (decreased CA/A ratio), but also potentiating the action of nitric oxide in the posterior pole, relaxing nonvascular smooth muscle (MFs) and improving hysteresis of the eye wall as a whole. The association of both drugs (Pgs and miotics) for treatment and cure of myopia has never been thought in the literature.

### STATE OF THE ART

Some prior art documents describe options for the treatment of myopia using some drugs alone or in combination, for example:

The document entitled “Pharmaceutical intervention for myopia control” (Ganesan et al.) is a broad and well-updated review of the pharmacological treatments available/under study for the treatment of myopia and serves as an excellent reference for clarification on everything that has been discussed in the ophthalmic environment regarding the use of drugs for controlling myopia. It is possible to draw, from this document, important conclusions about the current state of the subject, mainly from the practical point of view, in ophthalmological offices, for example:

Currently, there are no effective drugs for the prevention and control of myopia. It is an extremely serious fact, if we are to link it to the data of the current epidemic of myopia that spreads across five continents.

Medicines such as Pirenzepine and atropine (diluted at the concentration of 0.01%) are currently the only therapeutic options (eye drops) against the progression of myopia. They are mydriatic drugs, which block cholinergic receptors (action exactly opposite to that of the miotics used in the present invention). They are drugs with extensive side effects precisely by keeping the eye dilated, exposing the retina and crystalline to the action of UV rays, which can damage the internal ocular tissues. They are also cycloplegic, which block accommodation, interfering in the student's daily life. For these reasons, they are medicines found in a few manipulation pharmacies, that is, they are rarely prescribed.

The document also alludes to the use of organophosphate insecticides (irreversible anticholinesterase substances) through intravitreal injection in laboratory animals. In addition, these potent anticholinesterase inhibitors bind the acetylcholinesterase enzyme irreversibly and induce spasm of accommodation and myopia, exactly the opposite of that is proposed by the present invention. The objective of the experiments was to observe the action of these drugs in the accidental poisoning of children and their effects on the evolution of myopia: the results were inconclusive. In the present invention, the serious side effects of irreversible anticholinesterases have been highlighted, which in the past was a relevant fact to no longer be prescribed in ophthalmology.

The nicotinic cholinergic cited in the document actually induce myopia and were used in laboratory animals. They are not part of the class of miotic cholinergic drugs either, which are agents used in the present invention.

The reversible anticholinesterase agents, main drug used in the present invention, were not mentioned at any point in this document.

Document PI0210013-4 discloses the use of drugs, such as pirenzepine, in the pharmacological treatment of myopia. However, such a substance is known to be a mydriatic rather than a miotic drug. This alone is enough to distance such document from the present invention.

Document CN105998030 discloses the use of prostaglandin F2α in the treatment of myopia and its effect on axial stretching of the eye. In addition, it mentions its concomitant use with dopamine. Although prostaglandin F2α (latanoprost) used in the preparation (with dopamine) is the same as used in the present invention, its technical description is wholly different from the one proposed in this document. In this document, its action is as an ocular hypotensive, inhibiting ocular stretching. This, alone, is not innovation. In addition, said document cites a special form of deprivation myopia, which is ocular stretching in laboratory animals. In humans, deprivation myopia should be less than 1% of the total cases. The association with dopamine for this purpose does not prevent or treat myopia. In the present invention, the action of prostaglandins is to potentiate the action of miotics in the posterior pole. Another detail is that, in the present invention, prostaglandin is of optional use.

Document WO2009/077736 refers to the use of pilocarpine, a miotic substance, to improve visual acuity in a variety of conditions, including myopia. Nevertheless, it is emphasized that in said document, the use of pilocarpine is concomitant with the use of a sympathetic system antagonist. The document itself begins as an optical correction. Said pilocarpine is a miotic, known as a drug that causes spasm of accommodation and high-grade myopia. This is exactly the opposite of what is proposed in the present invention, which is prevention and treatment of myopia. In said document, the optical action of pilocarpine is in the formation of a pinhole, an optical mechanism well known for its use in presbyopia. It is also used in an association of sympathetic blocking drugs. There is no technical description for the drug action.

Therefore, in view of the state of the art, the present invention unexpectedly proposes the use of miotic cholinergic substances alone or in combination with prostaglandin F2α analogues, for the preparation of a medicament compound for the prevention and treatment of myopia, by decreasing the Accommodative Convergence/Accommodation (AC/A) Ratio, which is elevated at the onset and progression of myopia, thus being responsible for the genesis of myopia.

### SUMMARY OF THE INVENTION

The present invention has the object of proposing a novel and unprecedented use of miotic substances, alone or in combination with prostaglandin F2α analogs in the formulation of eye drops for topical use of ocular substances to prevent or inhibit the progression of myopia, as well as its reduction or treatment in some cases, and such medicinal compounds may be also used postoperatively for myopia surgeries to prevent their relapse. The novel use is based on the action of the parasympathetic-nitrergic system in reducing the AC/A ratio, which is abnormally high in myopia.

### BRIEF DESCRIPTION OF THE FIGURES

In order to obtain a complete and full overview of the object of this invention, reference figures are presented, as follows.

FIG. 1 shows the difference between the classic ocular accommodation mechanism, supported by Helmholtz and the most current one presented in the present invention.

FIG. 2 shows the graphs of the CA/A ratio over a period of 10 years, showing in (A) the difference between the various ethnicities and in (B) the difference between emmetropic and myopic individuals;

FIG. 3 shows the graph of the AC/A response ratio with the refractive category;

FIG. 4 shows the local tissue hysteresis graph;

FIG. 5 shows the elevated vitreous pressure graph (IVP);

FIG. 6 presents the three groups of NVSM that can be distinguished in the posterior wall of the adult eyes, distributed in the various layers of the sclera and choroid.

FIG. 7 shows a sequence of chemical reactions showing nitric oxide (NO) formed by the vascular endothelial cell that activates the guanylate cyclase enzyme producing increased levels of cyclic GMP (cGMP). In a cascade of reactions, Ca²⁺-dependent K⁺ channels open in the vascular smooth muscle cells (or in the cavernous body of the penis, or in the non-vascular muscles of the choroid) promoting muscle relaxation.

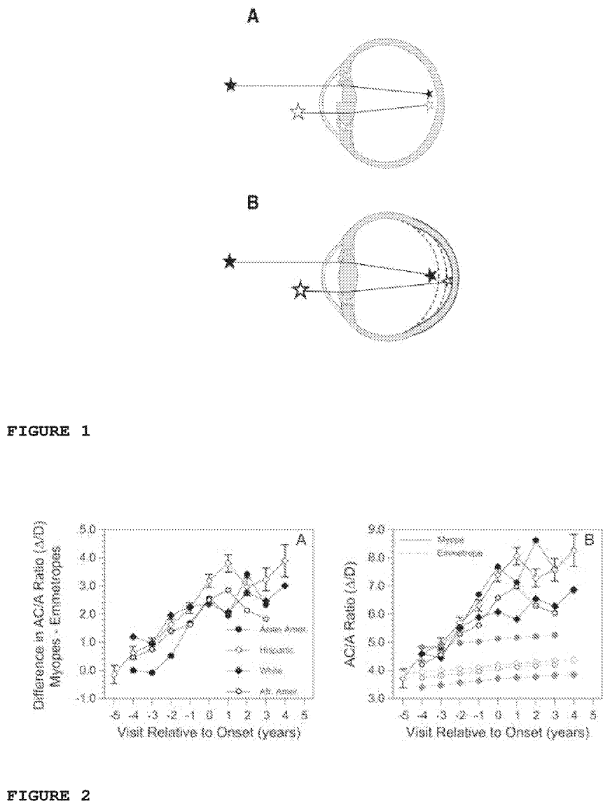

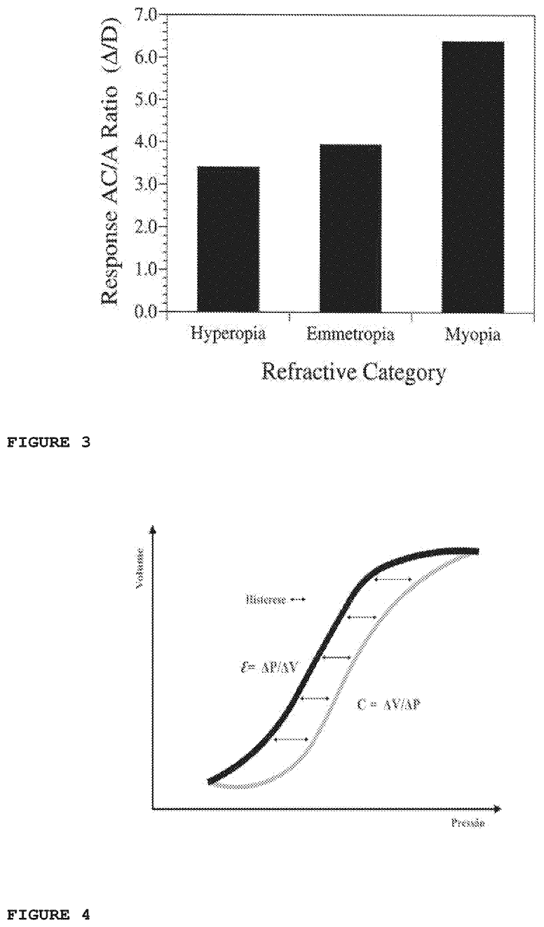

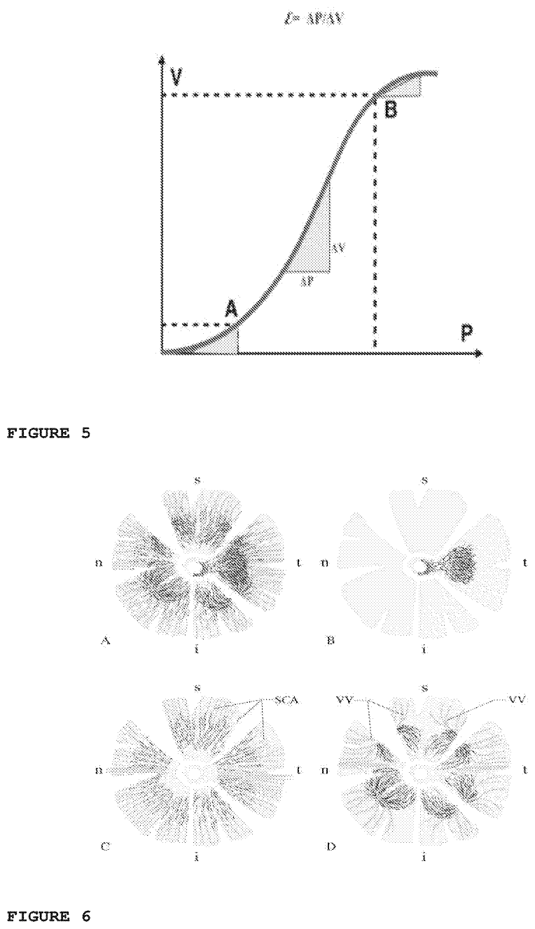

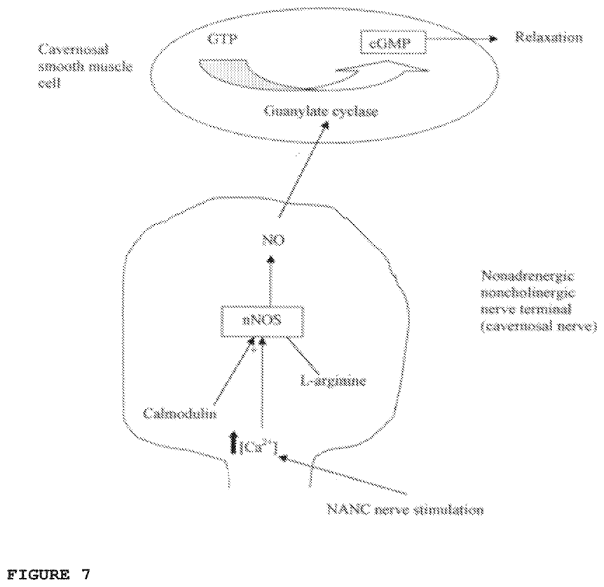

### DETAILED DESCRIPTION OF THE INVENTION

The present invention relates to the use of miotic cholinergic substances alone or in combination with prostaglandin F2α analogues, for the preparation of a medicament compound for the prevention and treatment of myopia, by decreasing the Accommodative Convergence/Accommodation Ratio (AC/A), which is elevated at the onset and progression of myopia, thus being responsible for the genesis of myopia.

As will be appreciated, the present invention will provide improved visual acuity for all distances, with consequent improvement in contrast sensitivity and image sharpness, increased choroidal irrigation, and improved retinal cell action in the image receiving process.

The parasympathetic system is the great conductor of ocular physiology, as has been mentioned so far. The literature findings of the last years showed the direct interference of the oculomotor nerve in the accommodation, in the emmetropization and mainly in the genesis of myopia. This fact is not surprising, as dozens of researches in the field have shown an intimate relationship between long-term and sustained nearwork for several years and the onset of myopia in young people of all races and both sexes.

Many researches have focused on the search for genes that were directly or indirectly linked to the genesis of myopia. In this sense, many genetic markers were related to cases of family heredity of myopia. However, gene therapy is still very far from the current stage of medical development. The most surprising finding in these studies was the emergence of a novel biological marker for myopia, in this case, the AC/A ratio, which was already studied in ophthalmology in the last century, but was never associated with myopia.

Until the early 1980s, this measure assisted in the diagnosis of accommodative esotropia and in the treatment of these ocular deviations. Thus, irreversible (very potent) anticholinesterases were used to reduce abnormally high AC/A ratios to regularize strabismus without the use of corrective lenses. The use of these substances was purely empirical, since their true action in reducing AC/A was not known. In fact, the ocular physiology involved in the formation of this ratio was also unknown.

The advantage of using potent anticholinesterase drugs with eye drops was mainly the time of action (half-life around 24 hours), for the convenience to the parents, since it needed continuous use for several years. However, the association of these drugs with several side effects avoid their prescription by ophthalmologists, which increasingly opt for the use of glasses.

Site and Mechanism of Action

Miotics (including acetylcholinesterase inhibitors and other cholinergic drugs such as pilocarpine) have been widely prescribed in the last century (and are still used) as ocular antihypertensive drugs in glaucomatous patients. They are moderate-acting drugs to reduce IOP and have several undesirable side effects to be used chronically. The main one was the spasm of accommodation and myopization of young patients.

Thus, one of the differences of the present invention is the use of a miotic to prevent exactly what was thought to be a complication of the chronic use of these substances: myopia. The first justification for explaining such action is the concentration of these drugs, much more diluted in the use proposed for the present invention. In addition, the site and mechanism of action involved would justify the apparent innovative paradox of these drugs in the treatment of myopia.

The miotics act in several sites of the eyeball, according to the parasympathetic innervation of the oculomotor nerve. In the history of the present invention, two forms of action of muscarinic receptors (primarily M3) have been described, either by contracting the ciliary or sphincter muscle of the pupil (known as the traditional pathway), or by relaxing the smooth muscle of blood vessels and nonvascular smooth muscle, by nitrergic-parasympathetic action (via activation by nitric oxide).

The miotics act on two sites in the parasympathetic ocular system; one on the posterior pole, relaxing the smooth muscle of the choroid (nitrergic pathway) and improving the elastance, reducing AC/A ratio and combating myopia. The other action is by the traditional route, contracting the ciliary and sphincter muscles of the pupil, increasing drainage of aqueous humor by the trabecular (intermediate portion of the eye) and reducing IOP. Therefore, justifying a different action of miotics in the prevention of myopia.

These different sites of action of miotics could also justify the spasm of accommodation observed at high concentrations of these substances. At high concentrations, the action of contraction of the ciliary muscle and the sphincter muscle of the pupil would overlap with the relaxation action of the posterior pole, resulting in eye elongation and myopization.

In addition, another important differential in the present invention is the action of miotic cholinergic drugs as a chemical substitute for solar irradiation (UV) on the posterior pole of the eyeball (reduced in cases of myopia). UV radiation inactivates the enzyme acetylcholinesterase. Thus, under the action of these drugs, there is a greater availability of acetylcholine, with consequent improvement of the elasticity of the posterior pole and a protective effect on the development of myopia.

Considering everything that has been discussed so far, close focusing in the absence of UV light are the two main causes of myopia; cholinergic miotic agents acts directly on both.

Action of F2α Prostaglandins

Prostaglandins (Pgs) are potent ocular hypotensive agents and will be used in the present invention as adjuvant substances in the prevention of myopia. The exact mechanism of action of Pgs as a hypotensive drug is still controversial, but probably facilitates the removal of the aqueous humor by the supracoroidal pathway. In other sites of the organism, the Pgs are considered vasodilators and could have this action on the choroid, potentiating the action of the miotics on the smooth muscle of the choroid and improving the elastance of the posterior wall of the eyeball.

Thus, as hypotensives, Pgs and miotics would act on the intermediate portion of the eye. In the prevention and treatment of myopia, these drugs would act in the posterior pole.

Clinical Trials

After finding that the miotic cholinergic substances in combination with the F2α prostaglandin analogs (currently used as ocular hypotensives drugs for treating glaucoma) also act together and effectively in the prevention and treatment of myopia by reducing the AC/A ratio (elastance of the posterior pole) and ocular stretching, the present invention proposes the use of these substances in myopic patients or in patients with night myopia (tonic accommodation). It was noted that, in some cases, myopia would not only fail to progress, but also regress. These drugs were well accepted for both adults and children and teenagers, with minimal side effects. The periodic evaluation of the AC/A ratio is an extremely useful parameter in the clinical follow-up of these patients. In the long term, it has also been found that the chronic reduction of AC/A ratio and tonic accommodation is an essential factor in the genesis of myopia and, therefore, capable of preventing its evolution, as previously described. Thus, the present invention proposes that it is possible to prevent, halt the evolution and even reverse myopia by means of medicinal treatment based on the use of miotic cholinergic substances in combination with F2α prostaglandin analogues (ocular, topical), bringing a new perspective to this field.

Applied Methodology

The methodology applied in the present invention is intended to demonstrate efficacy in the use of miotic cholinergic substances alone or in combination with F2α prostaglandin analogues for the prevention and treatment of myopia; and is divided into 5 phases:

1^{st} Phase:

Firstly, a clinical evaluation of the patient, visual acuity for distance and near, complementary ophthalmologic examinations, investigation of systemic diseases (hypertension), smoking, alcoholism and medication of local or systemic use is carried out. It is also important to investigate reading habits, computer use and the investigation of the occurrence of myopia in the family (hereditary antecedents) and previous ocular surgeries.

2^{nd} Phase:

Subsequently, an evaluation of the intraocular pressure (IOP), the retina, the extrinsic motility and the AC/A ratio is made.

3^{rd} Phase:

Subsequently, miotic cholinergic substances (the substance with minimal side effects, when administered in low concentrations, with emphasis on neostigmine, physostigmine, pyridostigmine and carbachol) are administered in liquid form in varying concentrations (from 0.0001% to 4.0%), applying 1 to 10 drops periodically (every 12 hours up to every 48 hours) in each eye.

The visual acuity is then periodically reevaluated for far and near distances, with refraction under cycloplegia. If myopic graduation remains unstable and elevated, prostaglandin analogues (a substance with minimal side effects, especially latanoprost, bimataprost, and travaprost) are associated in liquid form in varying concentrations (from 0.0001% to 0.5%), applying 1 to 10 drops periodically (every 12 hours up to every 48) in each eye.

4^{th} Phase:

Together with the use of the medications described above, patients should be guided on some practical measures that reduce the nearwork of the ciliary muscle, helping to contain the myopization process:

(i) Do not use myopia corrective lenses for near vision, especially for a long time (reading, computer use, etc.).

(ii) In case of need for prolonged use of near vision (excess accommodation), intercalate periods of ocular rest, with outdoor activities and sun exposure.

(iii) In case of prolonged use of near vision, move away as much as possible from the focus, decreasing the accommodation stimulus and inhibiting myopia development.

(iv) Undercorrection of the myopic error, bilaterally, or in the form of monovision, avoiding the “defocus” phenomenon.

5^{th} Phase:

With the stabilization of myopia, one can choose (or not) the gradual withdrawal of medicines, with periodic revision of the refraction degree.

In addition, the present invention claims that the current myopia epidemic is an iatrogenic effect due to myopic hypercorrection in the prescriptions generated in refractive consultations around the world. In order to provide the best visual acuity to myopic patients, ophthalmologists and optometrists hypercorrect the myopic error related to tonic accommodation, which can be counteracted by the use of miotics alone or in combination with F2α prostaglandins.

According to the present invention, the use of miotic cholinergic substances (alone or in combination with and F2α prostaglandin analogues) for the treatment and prevention of myopia would have absolute indication for the following groups of patients:

(i) Children who have early myopia of medium degree (1 to 5 degrees), with a high risk of developing pathological myopia or myopia in progression. The present invention also found that, in several cases, there is a significant reduction of myopic grade (tonic accommodation), which would be a stimulus for continuing with the treatment by using such substances.

(ii) Teenagers with juvenile myopia triggered mainly by nearwork, who present nocturnal myopia (also known as tonic accommodation), which can range from −0.50 to −3.00 SD. The present invention has found that such teenagers may have reduced or even eliminated degree of myopia (emmetropia).

(iii) Adults with late adult onset myopia triggered mainly by nearwork, who present night myopia (also known as tonic accommodation), which can range from −0.50 to −3.00 SD. The present invention has found that such individuals may have reduced or even eliminated degree of myopia (emmetropia).

(iv) Teenagers and adults who have myopia of medium degree (1 to 5 degrees), with a high risk of developing pathological myopia or myopia in progression. The present invention also found that, in several cases, there is a significant reduction of myopic grade, which would be a stimulus for continuing with the treatment by using such substances.

(v) Currently, safer surgical techniques (PRK and LASIK) impose limits on myopic graduation for its indication and are, therefore, contraindicated for cases of high myopia (above 9 degrees). The early reduction of the myopic grade in teenagers (by the drug treatment proposed in the present invention) makes possible the surgical indication with greater safety and for a greater number of myopic young people. Continuous use of these drugs may abbreviate the indication for refractive surgeries since the degrees would be stabilized or reduced during their use, facilitating surgical indication.

(vi) Myopic patients undergoing refractive surgery may present with recurrence of myopia and the use of these topical medications may completely correct this myopic residual, avoiding a surgical retouch.

(vii) Adults with late evolution myopia, who present an unstable graduation due to excessive nearwork (persistent computer users, readers, etc.). Such individuals have a higher incidence of glaucoma, cataract and retinal detachment. In the same way as teenagers, their myopic degree can be stabilized, avoiding the complications described above, besides enabling the early surgical indication. After surgery, such individuals should maintain the drug treatment (proposed by the present invention) to prevent relapse of myopia.

(viii) Individuals of all ages, with difficult to control myopia and with high AC/A ratio, with a tendency to develop pathological myopia, or even worsening of lesions due to pathological myopia. Pathological myopia is one of the major causes of blindness in the United States, occurring in abnormally elongated eyes, with a refractive error generally exceeding 6 degrees of myopia (25.5 mm). It causes tissue damages and high incidence of retinal detachment. Therefore, individuals with potential propensity to develop pathological myopia should maintain the drug treatment (proposed by the present invention) throughout life.

Although the invention has been widely described, one person skilled in the art would find obvious that many changes and modifications may be made to improve the project without covering said modifications by the scope of the invention.

### BIBLIOGRAPHIC REFERENCES

Dolgin E. The myopia boom. Nature. 2015; 519(7543):276-8.

Holden et al.: Global prevalence of myopia and high myopia and temporal trends from 2000 through 2050 (Ophthalmology 2016; 123:1036-1042)

Von Helmholtz H. Physiological Optics. New York: Dover, 1962; vol. I, 143-172, 375-415

Santos-Neto E, Alves M R: New concepts in accommodation and presbyopia Rev Bras Oftalmol. 2011; 70 (5): 306-11

Prepas S B. Light, literacy and the absence of ultraviolet radiation in the development of myopia. Med Hypotheses. 2008 70(3):635-7.

W H Bishop, L Henke, J P Christopher, D B Millar. Photodestruction of acetylcholinesterase. Proc Natl Acad Sci USA. 1980 April; 77(4):1980-2.

Bayramlar H, Cekiç O, and Hep [symbol] en I F: Does convergence, not accommodation, cause axial-length elongation at near? A biometric study in teens. Ophthalmic Res. 1999; 31(4): 304-8

Koretz J F, Handelman G H. A model for accommodation in the young human eye: the effects of lens elastic anisotropy on the mechanism. Vision Res. 1983; 23(12): 1679-86

Brown P N, Koretz J F, Bron A J. The development and maintenance of emmetropia. Eye. 1999; 13(1): 83-92.

Mutti D O, e cols. CLEERE Study Group. The Response AC/A Ratio Before and After the Onset of Myopia. Invest Ophthalmol Vis Sci. 2017; 58(3): 1594-1602

Mutti D O, Jones L A, Moeschberger M L, Zadnik K. AC/A ratio, age, and refractive error in children. Am J Ophthalmol. 2000; 130(5): 690.

Flom M C, Takahashi E. The AC/A ratio and undercorrected myopia. Am J Optom Arch Am Acad Optom. 1962; 39: 305-b 312.

Van Alphen G. On emmetropia and ametropia. Opt Acta (Lond). 1961; 142: (Suppl): 1-92.

May C A. Non-vascular smooth muscle cells in the human choroid: distribution, development and further characterization. J Anat. 2005; 207(4): 381-90.

Faraci F M, Brian J E Jr. Nitric oxide and the cerebral circulation. Stroke. 1994; 25(3): 692-703.

H J C Chen, G Spiers, C Sernia, and N A Lavidis. Response of the nitrergic system to activation of the neuroendocrine stress axis. Front Neurosci. 2015; 9: 3.

1. Use of miotic cholinergic substances, CHARACTERIZED in that it is for the manufacture of a medicament for the prevention and treatment of myopia.

2. Use, according to claim 1, CHARACTERIZED in that it is by the activation of the parasympathetic-nitrergic system.

3. Use, according to claim 1, CHARACTERIZED in that it is optionally combined with F2α prostaglandin analogues, depending on the stabilization or reduction of the refraction degree.

4. Use, according to claim 1, CHARACTERIZED in that the miotic cholinergic substances are administered in liquid form in concentrations ranging from 0.0001% to 4.0%, applying from 1 to 10 drops periodically, every 12 hours up to every 48 hours in each eye.

5. Use, according to claim 4, CHARACTERIZED in that the miotic cholinergic substances comprise mainly neostigmine, physostigmine, pyridostigmine, edrophonium and rivastigmine.

6. Use, according to claim 5, CHARACTERIZED in that the miotic cholinergic substances act in a manner to replace sunlight deprivation in myopic individuals, acting directly (therefore) on the two most well-known causes of myopia: nearwork in the absence of UV light.

7. Use, according to claim 3, CHARACTERIZED in that the F2α prostaglandin analogues are administered in liquid form in concentrations ranging from 0.0001% to 0.5%, applying from 1 to 10 drops periodically, every 12 hours up to every 48 hours in each eye.

8. Use, according to claim 7, CHARACTERIZED in that the F2α prostaglandin analogues comprise mainly latanoprost, bimataprost, unoprostone and travaprost.

9. Use, according to any one of claims 1 to 8, CHARACTERIZED in that it stabilizes and reduces the refraction degree.

10. Use, according to any one of claims 1 to 8, CHARACTERIZED in that it reduces the elastance of the posterior pole of the eye and, consequently, the AC/A ratio.

11. Use, according to any one of claims 1 to 8, CHARACTERIZED in that it improves visual acuity for all distances, with consequent increase of the sensitivity of contrast and sharpness of the image, by the greater irrigation of the choroid and better action of the retinal cells in the process of reception of the image.

12. Use, according to any one of claims 1 to 11, CHARACTERIZED in that it is indicated for children who have early myopia of medium degree (1 to 5 degrees), with a high risk of developing pathological myopia or myopia in progression.

13. Use according to any one of claims 1 to 11, CHARACTERIZED in that it is indicated for teenagers with juvenile myopia triggered mainly by nearwork, who present night myopia ranging from −0.50 to −3.00 DE.

14. Use according to any one of claims 1 to 11, CHARACTERIZED in that it is indicated for adults with late-onset myopia, triggered mainly by nearwork, who present night myopia ranging from −0.50 to −3.00 DE.

15. Use, according to any one of claims 1 to 11, CHARACTERIZED in that it is indicated for teenagers and adults who have myopia of medium degree (1 to 5 degrees), with a high risk of developing pathological myopia or myopia in progression.

16. Use, according to any one of claims 1 to 11, CHARACTERIZED in that it is indicated for cases of high myopia (above 9 degrees), in which surgeries are contraindicated.

17. Use, according to any one of claims 1 to 11, CHARACTERIZED in that it is indicated for myopic patients undergoing refractive surgery.

18. Use according to any one of claims 1 to 11, CHARACTERIZED in that it is indicated for adults with late evolution myopia, who exhibit unstable graduation due to excess of nearwork.

19. Use, according to any one of claims 1 to 11, CHARACTERIZED in that it is indicated for individuals of all ages, with difficult to control myopia and with high AC/A ratio, tending to develop pathological myopia, or even worsening of lesions due to pathological myopia.
